(19) United States
(12) Patent Application Publication
(10) Pub. No.: US 2016/0030652 A1
(43) Pub. Date: Feb. 4, 2016

(54) Implantable Cable-Connecting Device

(71) Applicant: BERLIN HEART GMBH, Berlin (DE)

(72) Inventors: Andreas ARNDT, Berlin (DE); Peter NÜSSER, Kleinmachnow (DE); Kim Peter WINTERWERBER, Berlin (DE)

(21) Appl. No.: 14/782,245

(22) PCT Filed: Apr. 4, 2014
(86) PCT No.: PCT/EP2014/056775

§ 371 Date: Oct. 2, 2015

Related U.S. Application Data
(60) Provisional application No. 61/808,301, filed on Apr. 4, 2013.

(30) Foreign Application Priority Data
Apr. 4, 2013 (EP) 13162255.7

(51) Int. Cl. A61M 1/12

(52) U.S. Cl. CPC A61M 1/127; A61M 1/122; A61M 2205/8262; A61M 2205/3523; A61M 2205/04

(57) ABSTRACT

An implantable cable connection device is provided with a cable connector which can comprise several plug-in connection elements, to which in each case an implantable cable can be connected by way of a plug-in connection, where the cable connector is designed as a rigid body of a biocompatible material.

### CROSS-REFERENCE TO RELATED APPLICATIONS

This application is a 371 nationalization of PCT/EP2014/056775, entitled “IMPLANTABLE CABLE-CONNECTING DEVICE,” having an international filing date of Apr. 4, 2014, the entire contents of which are hereby incorporated by reference, which in turn claims priority to U.S. provisional patent application 61/808,301 filed on Apr. 4, 2013, entitled “IMPLANTABLE CABLE CONNECTING APPARATUS,” the entire contents of which are hereby incorporated by reference, and to European patent application 13162255.7 filed Apr. 4, 2013, entitled “Implantable cable connection device”, the entire contents of which are hereby incorporated by reference.

### BACKGROUND

The invention lies in the field of mechanics and electrotechnology and can particularly advantageously be applied in medical technology. The invention especially relates to a cable connection device which is implantable.

Different active implants which partially replace or supplement body parts, are common in medical technology. The often comprise sensors or actuators and can realise mechanical or electric functioning manners. Many such implants are provided with electrical devices, for example sensors or also motors or control devices, which, for example for the purpose of energy supply or communication, are to be connected by way of percutaneous cables to the region outside the patient's body, into which they are implanted.

Such percutaneous cables, i.e. cables which in a region of their course are arranged within the patient's body and in another region of their course are arranged outside the patient's body, basically represent a risk concerning infection and inflammation. It is therefore desirable for a respective cable to be able to be removed and/or replaced in a simple manner, given the occurrence of complications. For this purpose, it is basically known to provide a plug-in (insert) connection within the body of a patient, in the case of an implantable cable.

For this purpose, a cable connector is known from the international patent application WO 2011/156391 A2, and this is designed as a so-called Y-cable, i.e. with a plug-in (insert) connector and a cable which is unreleasably fastened on this and is with several leads branching to two separate cables, wherein each of the separate cables again has its own plug-in connector. According to the mentioned document, such a cable connector is provided for the electrical connection of a percutaneous cable to two implantable pumps. These pumps serve for delivering blood in the blood circulation system.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates, in an overview representation, a cable connection device with two blood pumps and with a percutaneous cable in the implanted condition;

FIG. 2 illustrates a cable connector in cross section, with three plug-in connection elements;

FIG. 3 illustrates a cable connector in cross section, with two plug-in connection elements and a plane front surface;

FIG. 4 illustrates a cable connector with an arcuate front surface and two plug-in connection elements;

FIG. 5 illustrates a cable connector with a more detailed representation of the internal guiding of the leads;

FIG. 6 illustrates a locking device for two plug-in connections, on a cable connector; and

FIG. 7 to FIG. 12 illustrate various scenarios for placing in each case two heart assist pumps for the heart ventricle and a cable connector.

### DETAILED DESCRIPTION

Against the background the state of the art, it is the object of the present invention to create an implantable cable connector which is constructed in a particularly simple and stable manner and which is particularly simple to handle on implantation and on creating the plug-in connections.

The invention thereby relates to an implantable cable connection device with a cable connector which comprises two or more (preferably three or more) plug-in connection elements, to which an implantable cable can be connected in each case by way of a plug-in connection, wherein the cable connector is designed as a stiff body of a biocompatible material.

It is possible to implant the cable connector in a complete manner and to connect a percutaneous cable which creates the connection of the cable connector to the outside of the body, as well as a further, completely implanted cable and, as the case may be, further, in particular completely implanted cables, onto the cable connector, due to the arrangement of several plug-in connection elements on the cable connector. Due to the fact that the cable connector is designed as a stiff body, the entry and departure directions of the plug-in connection elements are reliably defined, and it is merely necessary to briefly or permanently fix the cable connector for creating and releasing the plug-in connections. The cable connector cannot deform on use, and the bringing of the inserted cable into an undesired position by way of deforming the cable connector is prevented or rendered more difficult on account of this. Moreover, the cable connector can be manufactured in a very compact manner as a stiff body and can be easily accommodated in the body of a patient.

One envisages arranging in particular three or more plug-in connection elements on the same side of the cable connector. Several cables can be stuck onto the plug-in connection elements of the cable connector, as is anatomically favourable, in particular without having to rotate this cable connector, on account of the geometry. This is particularly important if cables must be plugged in after the insertion of the cable connector into the patient's body, for example also with the exchange of defect cables.

A design envisages all plug-in connection elements being arranged on the same side of the cable connector. With this design of the cable connector, this can be implanted such that the plug-in connection elements in each case lie on the side of the cable connector which can be easily reached from the outside through a minimal opening of the patient's body, by way of minimal intervention. The cable connector itself then does not need to be removed from the patient's body for the exchange of the plug-in connections. The plug-in connection elements can advantageously be arranged on the smallest or narrowest of the side surfaces of the cable connector.

Expressed briefly, amongst other things, a cable connector is provided, with which all plug-in connection elements are on the same side of the cable connector and thereby three (or more) plug-in connection elements are accommodated on a front plate (which can be plane or arcuate, see reference numerals 21, 24, 24) of the cable connector. The plug-in ability of all cables is thus given from the same side, and the arrangement in particular is particularly applicable to an arrangement with two blood pumps.

The invention can furthermore be advantageously designed such that the plug-in axes of the plug-in connection elements arranged together on one side of the cable connector are aligned parallel to one another. The handling of the plug-in connections can also be simplified by way of a parallel, in particular stiff arrangement of the plug-in axes of the plug-in connection elements on the cable connector. The plug-in axes of the plug-in connection elements are to be understood in each case as the direction, in which a cable with the respective complementary plug-in connection element is stuck onto the plug-in connection element arranged on the cable connector. With common hermaphroditic plug-in connections, the plug-in axis is parallel to the longitudinal axis of the plug pins or the corresponding sockets.

A parallel alignment of the plug-in axes of the plug-in connection elements also leads to these being able to be designed with a minimal distance to one another, and to the corresponding complementary plug-in connection elements of cables likewise being able to be arranged in the same manner at a minimal distance to one another outside the cable connector. The total used and required constructional space of the cable connection device is thus minimised.

In a particularly advantageous form, the invention is moreover further released by way of the cable connector having a side designed as a plane surface, on which the plug-in connection elements are arranged and all other boundary (delimitation) surfaces of the cable connector being convexly rounded. Such a cable connector designed in a largely rounded manner at its outer sides can be implanted into the body of a patient without further ado, in particular without the danger of any particular loading of the tissue in the rounded regions of the cable connector. Neither are injuries to be feared in the case of a movement of the tissue, in which the cable connector is embedded.

One can moreover advantageously envisage the cable connector being designed in an essentially hemispherically rounded manner. The cable connector apart from a hemisphere can also assume the shape of a halved ellipsoid or another rounded body.

A flat form of the cable connector, with which the length and width are a multiple of the height, is also particularly advantageous, wherein the corners can be rounded. The basic shape can also be that of a cuboid.

A further advantageous embodiment of the invention envisages the cable connector being designed as a housing with at least one interior. A cable connector manufactured as a compact body on the one hand can connect the different plug-in connection elements to one another within a small space by way of the favourable internal leading of leads, and moreover can yet comprise an interior, in which for example an electric energy storage means/accumulator or an electrically active element, for example an electrical circuit, in particular a device for the wireless communication, can be advantageously arranged. For example, a transponder can also be arranged in the cable connector, and this permits an identification without an energy supply becoming necessary (radio-tag). The interior can also be filled with an adhesive, a cast mass or a fluid, but a cavity can however also remain free.

A control device for one or more blood pump motors can also be arranged in the interior for example. For this, a microcontroller or another programmable device can be provided there.

Moreover, one can additionally envisage the cable connector at its outer sides comprising exclusively metal and/or glass and/or ceramic, in order to create an embodiment of the invention which is particularly tissue-compatible. These materials are biocompatible to a high extent, i.e. compatible with tissue, and stable over the longer term, so that a cable connector comprising such a material can be implanted for the longer term. The outer surfaces are advantageously designed in a smooth manner.

A metal which is particularly advantageous in this context is titanium. The housing of the cable connector can thereby also comprise several parts which are joined together, in particular welded, bonded or cast to one another, in a gas-tight manner, to the outer side of the cable connector. A metal element of the cable connector can be melted with a glass part for example. The glass part for its part in turn can be melted with a ceramic in a gas-tight manner. A metal can also be connected to a ceramic or a glass in a gas-tight manner by way of a metal soldering. Common to all these connection techniques is the fact that they permit a reliable fixation with a simultaneously gas-tight sealing and a chemicals as well as physical strength.

A cable connector in particular if it is used together with one or more heart assist blood pumps, can be accommodated in the intermediate space between the myocardium which is then usually somewhat reduced in size, and the pericardium. A subcutaneous arrangement of the cable connector is also possible, and eyelets for example can be provided on these for sewing on the body tissue.

The invention furthermore relates to a cable connection device with a cable connector, with a first percutaneous cable which is inserted into this, and with at least one second cable inserted into the cable connector, as well as with an implant, in particular a blood pump, which is connected to the second cable. Moreover, yet a third cable connected to the cable connector by way of a plug-in connection can optionally be provided, as well as a further implant, in particular likewise an implanted blood pump, which is connected to this third cable.

The cable connection device potentially thus not only can comprise the cable connector with the plug-in connection devices, but also the respective cables inserted into these and blood pumps or alternative implants, which are connected to these. An inserted, percutaneous cable connected to the cable connector can also belong to the cable connection device.

The cable connection device thus as a whole can comprise a system of several implants, in particular pumps, which are connected by way of cable connections via a cable connector to a percutaneous cable which is simply accessible outside the body of a patient at a cable connection.

The cable connector, in particular its housing, can moreover comprise at least one, in particular two coupling elements, for example screw threads, bayonet receivers or likewise, for a fixation element, by way of which the connector can be easily manipulated, particularly also with a cable plug-in procedure.

An advantageous design of the invention moreover envisages at least two plug-in connection elements being provided next to one another on the outer side of the cable connector and moreover a locking device being provided, which, as long as one of the plug-in connections is released in each case, blocks the release of the other plug-in connection, wherein a locking element comprising two cover elements for the at least partial covering of a plug-in connection in each case is provided for this, wherein the locking element is movable between two end positions and a plug-in connection is blocked in each case in each of the end positions.

Such a locking device prevents two plug-in connections at the cable connector from being inadvertently simultaneously released, which can lead to cables being mixed up on reinserting. This is particularly undesirable and risky if two cable connections to two blood pumps are released, said blood pumps being arranged in different parts of a patient's heart or at different locations with respect to the heart, in its periphery or generally in the blood circulation system, and their mix-up could be harmful. A mechanical coding of the plug-in connectors for their differentiation can also be envisaged additionally or alternatively to this measure.

An advantageous method for implanting a cable connection device of the type according to the invention can moreover envisage the cable connector being arranged in the patient's body and at least one, in particular two or three implantable cables thereafter being connected to the cable connector by way of plug-in connections.

Further implantable elements, such as blood pumps which are to be connected to the cable connector, can advantageously be implanted already before the implantation of the cable connector or also after the implantation of the cable connector, and at all events they do not need to be introduced simultaneously with the cable connector.

The invention also relates to a method for the exchange of several implantable cables which are connected to a cable connector according to the invention, with which firstly the plug-in connection of a first cable is released, and a new first cable is connected to the cable connector by way of the plug-in connection, and thereupon the plug-in connection of a second cable is released and a plug-in connection of the cable connector to a new second cable is created, wherein in particular the first and the second cable are connected in each case to a blood pump. The mixing-up of two cables and/or plug-in connections is prevented by way of such a procedure.

The invention moreover also relates to a method for the exchange of an implanted cable which is connected to the cable connector according to the invention, with which firstly a holding tool is connected to a coupling element of the cable connector, thereupon the plug-in connection of the cable released, and a new cable is connected to the cable connector by way of a plug-in connection. In this manner, the cable connector as a stiff body can be firmly held from the outside whilst the plug-in connections are released or connected. The cable connector can thereby remain implanted, and only minimally comes into contact with instruments not belonging to the body.

A coupling element can basically have a mechanical fit, such as a screw thread or a bayonet closure, or it can also be magnetically formed, for example by way of a magnet fastened on the cable connector and/or on a holding tool.

An advantageous application of the cable connection device envisages the cable connection device being connected to two heart assist pumps and commonly arranged with these directly on a heart, in particular fixed on this or on the pericardium.

An advantageous method for implanting a cable connection device envisages the cable connector being electrically connected to two heart assist pumps and, temporally beforehand or thereafter, being fixed on the pericardium of a patient's heart.

The invention is hereinafter represented in a drawing and subsequently described, by way of one embodiment example. Thereby are shown in:

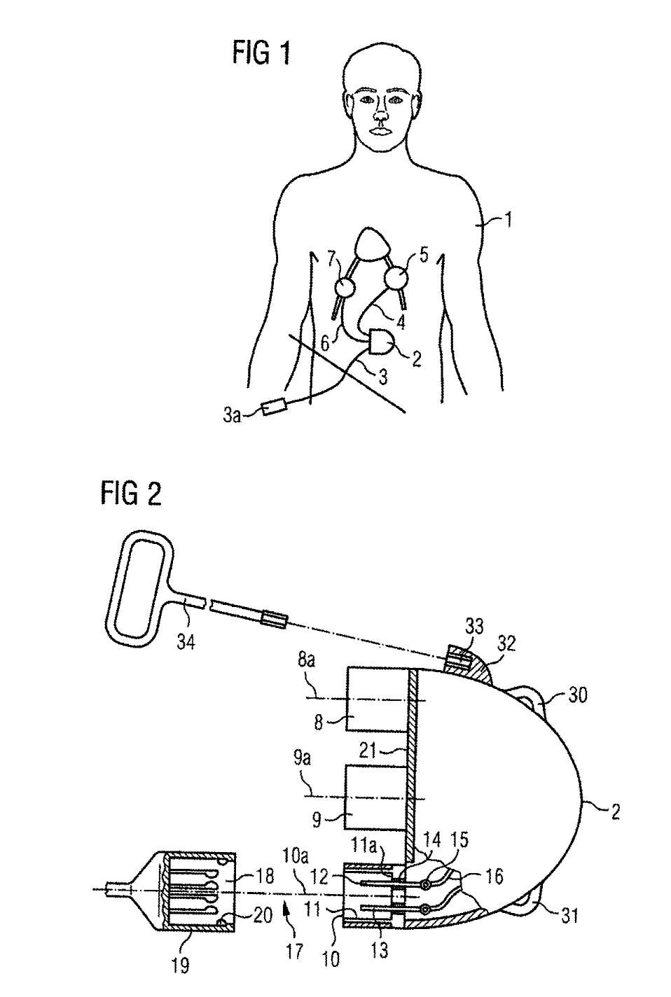

FIG. 1 in an overview representation, a cable connection device with two blood pumps and with a percutaneous cable in the implanted condition,

FIG. 2 a cable connector in cross section, with three plug-in connection elements,

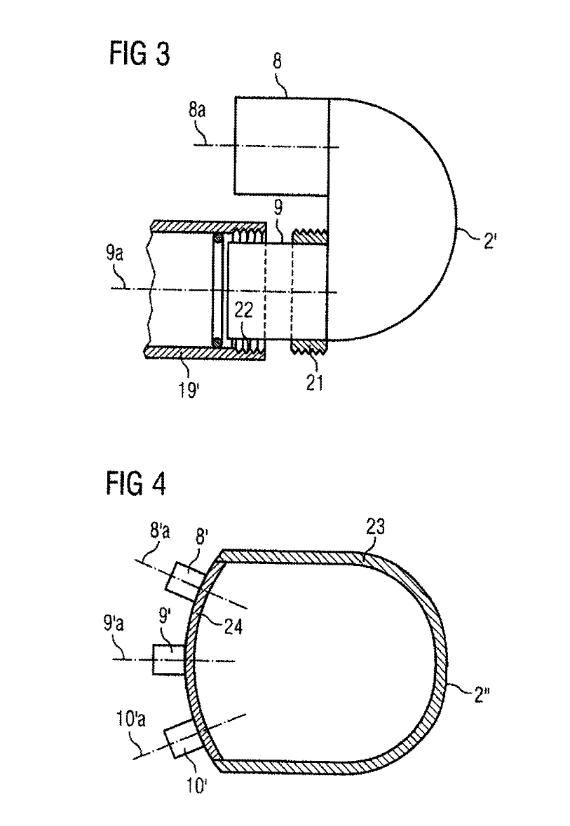

FIG. 3 a cable connector in cross section, with two plug-in connection elements and a plane front surface,

FIG. 4 a cable connector with an arcuate front surface and two plug-in connection elements,

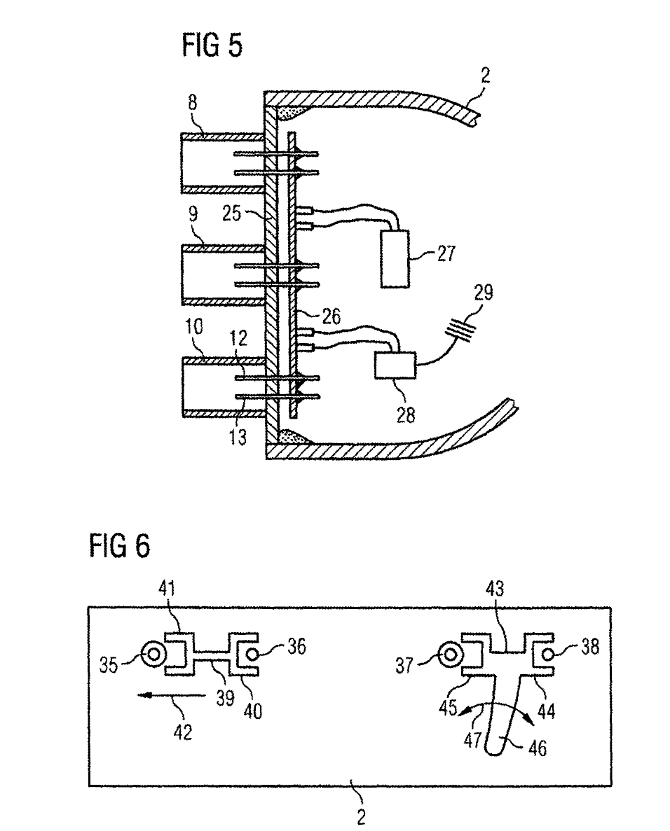

FIG. 5 a cable connector with a more detailed representation of the internal guiding of the leads,

FIG. 6 a locking device for two plug-in connections, on a cable connector, as well as

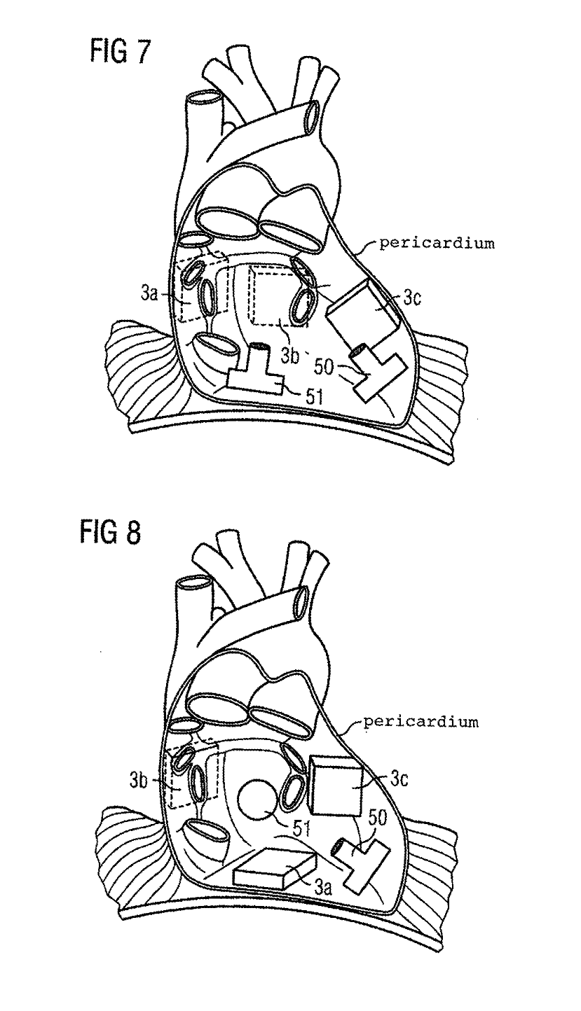

FIG. 7 to FIG. 12 various scenarios for placing in each case two heart assist pumps for the heart ventricle and a cable connector.

FIG. 1 schematically in a front view shows a patient's body 1, into which a cable connector 2 of a cable connection device is implanted. The cable connector 2 is connected to a first plug-in connection 3a outside the body of the patient, by way of a first implantable cable 3 which is designed as a percutaneous cable.

The term “percutaneous cable” usually indicates a cable which penetrates the skin or another part of the boundary surface between the body of the patient and the outside world. However, in the context of the present invention, a percutaneous cable can also indicate an implantable cable which runs completely within the patient's body and ends at a connection device, to which a cable leading further outside the patient's body can be coupled, for example inductively through the skin. One can also envisage only one plug-in connection 3a of the percutaneous cable being admitted into the skin and penetrating this.

A second implantable cable 4 which connects the cable connector 2 to a first blood pump 5 is moreover connected to the cable connector 2, apart from the percutaneous cable 3. A third implantable cable 6 on the one hand is connected to the cable connector 2 and on the other hand to a second blood pump 7. The implanted cables 3, 4, 6 are electrically connected to the cable connector 2 in each case via plug-in connections. The implantable cables at their end which is away from the cable connector 2 are advantageously connected in each case to a further element, for example to the blood pumps 5, 7, by way of a further plug-in connector 3a in each case. The implanted cables 4, 6 however can also be permanently and unreleasably connected to the respective blood pumps 5, 7.

The region of the skin which is required for leading through a cable, is opened and through which a foreign body permanently passes, is minimised due to the fact that several implanted elements, in the shown example the two blood pumps 5, 7 can be activated from outside the body of the patient via a single, percutaneous cable 3, on account of the cable connector 2. A particularly advantageous design results due to the fact that the percutaneous cable 3 is designed circularly in cross section, in order to minimise the contact surface between the cable and the skin of the patient's body as a whole and especially at the penetration location through the skin.

FIG. 2 schematically and partly in section shows a cable connector 2 in a detailed representation, wherein the cable connector 2 comprises three plug-in connection elements 8, 9 10. The individual plug-in connection elements on the cable connector 2 each have a hollow metal cylinder 11 which is fastened as a plug sleeve on the cable connector 2 and which comprises a cylinder base 11a which is connected to the cable connector 2 in a fluid-tight manner. The cylinder base 11a comprise recesses, through which plug pins 12, 13 are led in an electrically insulated and fluid-tight manner, and in a manner such that they are cast in the cylinder base 11a in a fluid-tight manner by way of a glass filling 14. The plug pins 12, 13, in the inside of the cable connector 2 where this has a cavity, are connected in each case via solder locations 15 to a lead, for example a copper core/strand 16 which for its part creates the contact of the plug pins 12, 13 to the other plug pins of the remaining plug-in connection elements 8, 9 or to electronic elements in the interior of the cable connector 2 or to an energy storage means in the interior of the cable connector 2.

The plug 17 which is stuck onto the metal cylinder 11 on the cable connector 2 comprises plug sleeves 18 which can be stuck onto the plug pins 1, 13, in order to create a galvanic contact to these. The plug sleeves 18 for their part are connected to leads which are led within the plug 17 to the connected, implantable cable. A plug housing 19 is provided, which has a shape which corresponds to the cylinder 11 and which can be stuck onto the cylinder 11. Thereby, an O-ring 20 of an elastomer which on the metal cylinder 11 seals to the outside in a fluid-tight manner is provided in the plug housing 19 in an inner peripheral groove.

A securing can also be provided which firmly holds the plug 17 on the cable connector 2, for example by way of a union nut which can be screwed on the cable connector 2 or by way of a bayonet connection between the plug 17 and the metal cylinder 11.

FIG. 2 shows that the cable connector 2 has a plane front surface 21, on which the plug-in connection elements 8, 9, 10 are fixed in a manner such that their plug-in axes 8a, 9a, 10a run parallel to one another.

The housing part of the cable connector 2 which connects onto the plane front plate forming the plane front surface 2 is designed for example as a spherical calotte or as part of a rotation ellipsoid and for example comprises metal, in particular titanium, or a ceramic. The connections between the metal and the ceramic can be designed in a fluid-tight manner by way of melting on the metal or by way of the intermediate application of a glass solder or metal solder. A fluid-tight pressing of the elements is conceivable by way of the applications of pressure.

Coupling elements, specifically holding eyelets 30, 31 are also represented by way of example in FIG. 2, as well as a housing lug 32, in which a threaded bore 33 is located, into which a holding tool 34 can be screwed for manipulation which is to say handling.

FIG. 3 shows a cable connector 2′ with only two plug-in connection elements 8, 9 on a plane front surface, wherein the plug-in connection elements 8, 9 with their respective plug-in axes 8a, 9a are aligned parallel to one another. An outer thread 21 on the metal cylinder is represented on the plug-in connection element 9, in the base region close to the front surface of the cable connector 2′, and this thread interacts with an inner thread 22 of a plug housing 19′ for fastening the plug-in connection.

FIG. 4 in comparison shows a cable connector 2″ with a rounded housing part 23 and with a front plate 24 which is likewise rounded, but thereby has a lesser rounding than the housing part 23. Three plug-in connection elements 8′, 9′, 10′ are fastened on the front plate 24 of the cable connector 2″. These, on account of the curvature of the front plate 24 are not parallel to one another with their plug-in axes 8′a, 9′a, 10′a, but are arranged at an angle between 10° and 30° to one another. Despite this, all plug-in elements can be inserted and released again from one side of the cable connector 2″.

A cable connector with plug-in connection elements 8, 9 10 is represented in FIG. 5, wherein there, plug pins 12, 13 pass through the front plate 25 of the cable connector in a gas-tight manner, for example sealed via melted glass. The plug-in pins 12, 13 are continued in the interior of the housing of the cable connector 2 and there pass through a circuit board 26. The plug pins 12, 13 are soldered to the circuit board 26 and to different ones of strip conductors which are located on this. Moreover, electrical or electronic elements as well as a strip conductor network can be provided on the circuit board 26, wherein the network or leading of the strip conductors, as desired, connects different plug pins of the cable connector 2 to one another or to an electronic component which is located in the interior of the cable connector 2. An electrical storage means in the form of an accumulator/battery 27 can also be provided there, and be connected to strip conductors of the circuit board 26.

An electronic device 28 which serves for the wireless communication between implants which are connected to the cable connector, and a device outside the patient's body can also be connected to the circuit board 26 via plug pins. For this purpose, the electronic device 28 is provided with an antenna 29 within the cable connector or a cavity in the cable connector 2.

The cable connection device according to the invention, with a cable connector and corresponding implanted cables, as well as, as the case may be, blood pumps or other elements, which are connected to these, serves for the secure operation of the implants, wherein cables can be replaced in a simple manner given the occurrence of errors in the connection technology, without implanted elements connected to the cable having to be necessarily removed or replaced.

FIG. 6 in a front view shows a cable connector 2 with plug-in connection elements 35, 36, 37, 38 in the form of plug pins which are each surrounded by plug-in sleeves and can be stuck onto the corresponding female cable plugs. The cable plugs in the plugged-in or inserted condition for example can be secured against inadvertent removal by way of a union nut or by way of a bayonet closure.

A locking element 39 is represented in the figure for the plug-in connection elements 35, 36 and this locking element comprises two cover elements 40, 41, of which the cover element 40 covers the plug-in connection element 36 in the shown position and blocks it by way of it preventing a griping/rotating of a stuck-on cable plug. If the locking element 39 is displaced to the left in the direction of the arrow 42, then the cover element 41 covers the plug-in connection element 35. Thus one of the plug-in connection elements 35, 36 is blocked in each case if the locking element 39 is pushed between the end positions, so that both plug-in connection are not released, at least not inadvertently. The locking element for example can be displaceably guided in a guide on the cable connector 2.

Alternatively, a locking element 43 is shown in the right part of the cable connector 2 in FIG. 6, and this locking element comprises two cover elements 44, 45 for the plug-in connection elements 37, 38 which are alternately covered and blocked on pivoting about the pivot axis 46 in the direction of the arrow 47. The locking elements 39, 43 can also be held in a bistable manner by way of a resilient toggle lever mechanism which is known per se, or another known means, so that they are stabilised in each case only in their end positions amid the covering of one plug-in connection element in each case.

A bus system which permits the addressing of a certain implant, in particular a blood pump, which is connected to the cable connector via a cable, independently of the plug-in connection element on the cable connector, in which the cable is inserted, can also be provided alternatively or additionally to a locking device.

A further implanted cable can yet be connected to the cable connector 2 by way of a plug-in connection, additionally to or instead of the cable 3 designed in a percutaneous manner and connected to the cable connector 2, and this further cable on the other hand is connected to an energy supply module or a communications module which is arranged directly below the skin of the patient and percutaneously permits an energy or information transmission via fields and/or waves. Basically, each cable connected to the cable connector, at its end which is away from the cable connector can be connected to one or several parallel, further leading leads in the context of a cascading.

The cable connection device which, as is illustrated in FIG. 1, comprises at least one percutaneous cable 3 and a fully implantable cable 4, is advantageously provided with cables of the following cable lengths, in particular if one or two implantable cables are connected to blood pumps:

Percutaneous cable: 100 to 160 cm length; first implantable connection cable to an implant: 20 to 40 cm length; as well as second implantable connection cable to a further implant: 20 to 40 cm.

FIGS. 7 to 12 each show advantageous further possibilities for the use of a cable connector in cooperation in each case with two heart assist pumps which each comprise a branch projecting into the left or the right heart ventricle. The cable connector is advantageously connected in each case to feed cables of the two pumps, which are connected to the cable connector by way of plug-in connections.

The cable connector can advantageously be arranged directly on the heart, for example in contact with the pericardium, advantageously also within the pericardium. This is particularly favourable if the heart is reduced in size in any case due to dysfunction.

The cable connector can further advantageously be fastened on the pericardium, for example fixedly sewn on thus.

The cable connector should thereby have a sufficient distance to the heart assist pumps.

The following possibilities for an optimised placement of the cable connector in dependence on the position of the heart assist pumps is hereafter represented, in accordance with the representations of FIGS. 7 to 10:

### FIG. 7: Placement of the Cable Connector and Biventricular Assist Device (Ventricular Assist Pumps for Both Ventricles): Scenario 1

1. LVAD (left ventricular assist device): inlet branch of the pump in the apex of the left ventricle (LV)
2. RVAD (right ventricular assist device): inlet branch in the inferior (diaphragmal) wall of the right ventricle (RV)
3. cable connector is positioned:
   a) between the right atrium (RA) and the pericardium
   b) between the anterior (free) wall of the RV and sternum
   c) between the lateral wall of the LV and pericardium

### FIG. 8: Placement of the Cable Connector and BVAD: Scenario 2

1. LVAD: inlet branch of the pump 50 in the apex of the left ventricle (LV)
2. RVAD: inlet branch of the pump 51 in the anterior (free) wall of the right ventricle (RV)
3. cable connector is positioned:
   a) between the inferior (diaphragmal) wall of the right ventricle (RV) and Pars
      diaphragmatica of the pericardium
   b) between the right atrium (RA) and the pericardium
   c) between the lateral wall of the LV and pericardium.

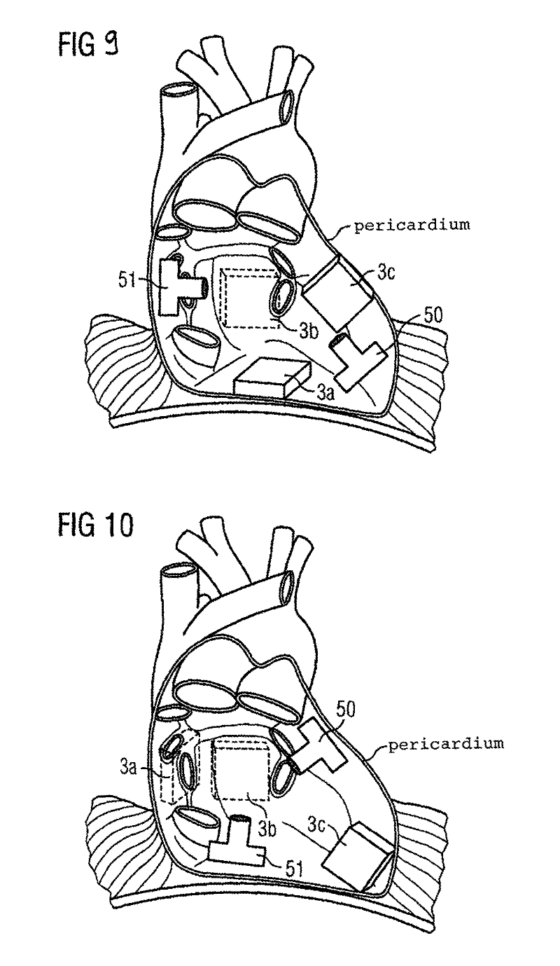

### FIG. 9: Placement of the Cable Connector and BVAD: Scenario 3

1. LVAD: inlet branch of the pump in the apex of the left ventricle (LV)
2. RVAD: inlet branch in the right atrium (RA)
3. cable connector is positioned:
   a) between the inferior (diaphragmal) wall of the right ventricle (RV) and Pars diaphragmatica of the pericardium
   b) between the anterior (free) wall of the RV and sternum
   c) between the lateral wall of the LV and pericardium.

### FIG. 10: Placement of the Cable Connector and BVAD: Scenario 4

1. LVAD: inlet branch of the pump in the left atrium (LA)
2. RVAD: inert branch in the inferior (diaphragmal) wall of the right ventricle (RV)
3. cable connector is positioned:
   a) between the right atrium (RA) and pericardium
   b) between the anterior (free) wall of the RV and the sternum
   c) between the apex of the left ventricle (LV) and the pericardium.

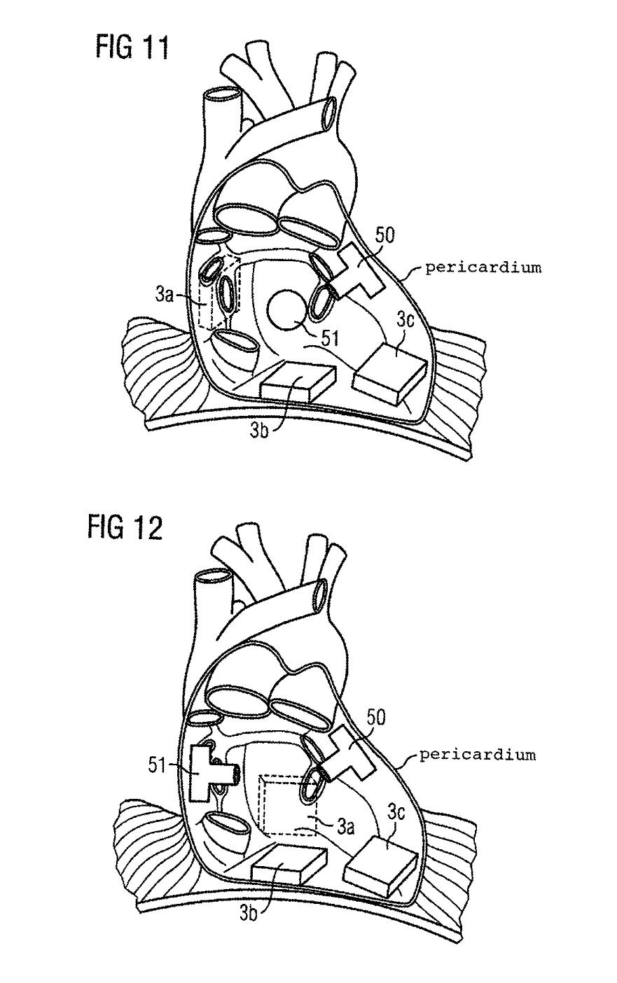

### FIG. 11: Placement of the Cable Connector and BVAD: Scenario 5

1. LVAD: inlet branch of the pump in the left atrium (LA)
2. RVAD: inlet branch in the anterior (free) wall of the right ventricle (RV)
3. cable connector is positioned:
   a) between the right atrium (RA) and the pericardium
   b) between the inferior (diaphragmal) wall of the RV and Pars diaphragmatica of the pericardium
   c) between the apex of the left ventricle (LV) and the pericardium.

### FIG. 12: Placement of the Cable Connector and BVAD: Scenario 6

1. LVAD: inlet branch of the pump in the left atrium (LA)
2. RVAD inlet branch in the right atrium (RA)
3. cable connector is positioned:
   a) between the anterior (free) wall of the right ventricle (RV) and the sternum
   b) between the inferior (diaphragmal) wall of the RV and the Pars diaphragmatica of the pericardium
   c) between the apex of the left ventricle (LV) and the pericardium.

All embodiment examples which are mentioned above, are described together or also independently of one another and can accordingly likewise be claimed in the form of patent claims. This relates to all aspects of the devices mentioned above, to their application as well as also to their implantation and manipulation in the human or animal body. This amongst other things relates to the aspects specified hereinafter:

1. An implantable cable connection device with a cable connector (2, 2′, 2″) which comprises two or more plug-in connection elements (8, 8′, 9, 9′, 10, 10′), to which an implantable cable (3, 4, 6) can be connected in each case by way of a plug-in connection, characterised in that the cable connector is designed as a stiff body of a biocompatible material.
2. A cable connection device according to aspect 1, characterised in that at least two, in particular three or more plug-in connection elements (8, 8′, 9, 9′, 10, 10′) are arranged on the same side of the cable connector (2, 2′, 2″).
3. A cable connection device according to aspect 1 or 2, characterised in that all plug-in connection elements (8, 8′, 9, 9′, 10, 10′) are arranged on the same side of the cable connector (21).
4. A cable connection device according to aspect 1, 2 or 3, characterised in that the plug-in axes (8a, 9a, 10a) of the plug-in connection elements which are arranged together on one side (21) of the cable connector, are aligned parallel to one another.
5 A cable connection device according to one of the aspects 1 to 4, characterised in that the cable connector (2, 2′, 2″) comprises a side (21) which is designed as a plane surface and on which the plug-in connection elements are arranged, and that all other boundary surfaces of the cable connector (2, 2′, 2″) are rounded in a convex manner.
6. A cable connection device according to aspect 5, characterised in that the cable connector (2, 2′, 2″) is designed rounded in an essentially hemispherical manner.
7. A cable connection device according to one of the aspects 1 to 6, characterised in that the cable connector (2, 2′, 2″) is designed as a housing (23) with at least one cavity.
8. A cable connection device according to aspect 7, characterised in that the housing (23) accommodates an electrical energy storage means (27) and/or an electrically active element (28, 29), in particular a device for wireless communication.
9. A cable connection device according to one of the aspects 1 to 8, characterised in that the cable connector (2, 2′, 2″) at its outer sides comprises exclusively metal and/or glass and/or ceramic.
10. A cable connection device according to one of the aspects 1 to 9, characterised in that the cable connector (2, 2′, 2″) comprises several parts which are welded and/or cast to one another in a gastight manner to the outer side of the cable connector.
11. A cable connection device according to one of the aspects 1 to 10, characterised by a cable connector (2, 2′, 2″), a first percutaneous cable (3) which is inserted into this, and at least one second cable (4) which is inserted into the cable connector, as well as a blood pump (5) connected to the second cable and in particular a third cable (6) inserted into the cable connector as well as a further blood pump (7) connected to the third cable.
12. The use of a cable connection device according to one of the aspects 1 to 11, characterised in that the cable connection device is connected to two heart assist pumps and together with these are arranged directly on a heart, in particular is fixed on this or on the pericardium.
13. A method for implanting a cable connection device according to one of the aspects 1 to 11, characterised in that the cable connector is electrically connected to two heart assist pumps, and is fixed on the pericardium of a patient's heart beforehand or afterwards with regard to time.
14. A cable connection device according to one of the aspects 1 to 11, characterised in that this comprises coupling elements for the releasable connection of holding tools.
15. A cable connection device according to aspect 14, characterised in that the coupling elements are designed as holding eyelets (30, 31) or as housing lugs (32), in which magnets, parts of a bayonet closure or a threaded bore (33) are located, into which a holding tool can be inserted for manipulation.
16. A cable connection device according to one of the aspects 1 to 11 or 14 to 15, characterised in that two adjacent plug-in connection elements have an alternating mechanical locking which is designed in a manner such that the plug-in connection elements cannot be simultaneously occupied with plugs.
17. A method for implanting a cable connection device, in particular according to one of the aspects 1 to 11, or 14 to 16, wherein the cable connector is arranged in the patient's body and thereafter at least one, in particular two or three implantable cables are connected to the cable connector by way of plug-in connections.
18. A method for exchanging several implantable cables which are connected to a cable connector according to one of the aspects 1 to 11 or 14 to 16, wherein firstly the plug-in connection of a first cable is released and a new first cable is connected to the cable connector by way of the plug-in connection, and thereupon the plug-in connection of a second cable is released and a plug-in connection of the cable connector to a new second cable is created, wherein in particular the first and the second cable are in each case connectable to a blood pump
19. A method for exchanging a cable which in particular is implanted with a cable connector according to one of the aspects 1 to 11 or 14 to 16, with which firstly a holding tool is connected to a coupling element of the cable connector, the plug-in connection of the cable is thereafter released and a new cable is connected to the cable connector by way of a plug-in connection.

1. An implantable cable connection device with a cable connector which comprises three or more plug-in connection elements, to which an implantable cable can be connected in each case by way of a plug-in connection, wherein the cable connector is designed as a stiff body of a biocompatible material, wherein all plug-in connection elements are arranged on the same side of the cable connector and thereby at least three plug-in connection elements are arranged on a front plate of the cable connector.

2. A cable connection device according to claim 1, wherein at least two, in particular three or more plug-in connection elements are arranged on the same side of the cable connector.

3. A cable connection device according to claim 1, wherein the plug-in axes of the plug-in connection elements which are arranged together on one side of the cable connector, are aligned parallel to one another.

4. A cable connection device according to claim 1, wherein the cable connector comprises a side which is designed as a plane surface and on which the plug-in connection elements are arranged, and that all remaining boundary surfaces of the cable connector are rounded in a convex manner.

5. A cable connection device according to claim 4, wherein the cable connector is designed rounded in an essentially hemispherical manner.

6. A cable connection device according to claim 1, wherein the cable connector is designed as a housing with at least one cavity.

7. A cable connection device according to claim 6, wherein the housing accommodates an electrical energy storage means and/or an electrically active element for wireless communication.

8. A cable connection device according to claim 1, wherein the cable connector at its outer sides comprises exclusively at least one of metal, glass, or ceramic.

9. A cable connection device according to claim 1, wherein the cable connector comprises several parts which are welded and/or cast to one another in a gas-tight manner to the outer side of the cable connector.

10. A cable connection device according to claim 1, wherein a first percutaneous cable is inserted into the cable connector, and at least one second cable is inserted into the cable connector, wherein a blood pump is connected to the second cable, a third cable is inserted into the cable connector, and a further blood pump is connected to the third cable.

11. The use of a cable connection device according to claim 1, wherein the cable connection device is connected to two heart assist pumps and together with the heart assist pumps is arranged directly on a heart, in particular is fixed on this or on the pericardium.

12. A method for implanting a cable connection device, the cable connection device comprising a cable connector which comprises at least three plug-in connection elements, wherein an implantable cable is connectable to any of the at least three plug-in connection elements by way of a plug-in connection, wherein the cable connector is designed as a stiff body of a biocompatible material, wherein all of the at least three plug-in connection elements are arranged on one side of the cable connector and thereby the at least three plug-in connection elements are arranged on a front plate of the cable connector, the method comprising: connecting the cable connector electrically to two heart assist pumps and fixing the cable connector on the pericardium of a patient's heart beforehand or afterwards with regard to time.

13. A method for implanting a cable connection device, the cable connection device comprising a cable connector which comprises at least three plug-in connection elements, wherein an implantable cable is connectable to any of the at least three plug-in connection elements by way of a plug-in connection, wherein the cable connector is designed as a stiff body of a biocompatible material, wherein all of the at least three plug-in connection elements are arranged on one side of the cable connector and thereby the at least three plug-in connection elements are arranged on a front plate of the cable connector, the method comprising arranging the cable connector in a patient's body and thereafter connecting at least one implantable cable to the cable connector by way of plug-in connections.

14. A method for exchanging several implantable cables which are connected to a cable connector, the cable connection device comprising a cable connector including at least three plug-in connection elements, wherein an implantable cable is connectable to any of the at least three plug-in connection elements by way of a plug-in connection, wherein the cable connector is designed as a stiff body of a biocompatible material, wherein all of the at least three plug-in connection elements are arranged on one side of the cable connector and thereby the at least three plug-in connection elements are arranged on a front plate of the cable connector, the method comprising firstly releasing the plug-in connection of a first cable and connecting a new first cable to the cable connector by way of the plug-in connection, and thereupon releasing the plug-in connection of a second cable and creating a plug-in connection of the cable connector to a new second cable, wherein the first and the second cables are in each case connectable to a blood pump

15. A method for exchanging a cable which in particular is implanted with a cable connector, the cable connection device comprising a cable connector including at least three plug-in connection elements, wherein an implantable cable is connectable to any of the at least three plug-in connection elements by way of a plug-in connection, wherein the cable connector is designed as a stiff body of a biocompatible material, wherein all of the at least three plug-in connection elements are arranged on one side of the cable connector and thereby the at least three plug-in connection elements are arranged on a front plate of the cable connector, the method comprising firstly connecting a holding tool to a coupling element of the cable connector, releasing the plug-in connection of the cable, and connecting a new cable is to the cable connector by way of the plug-in connection.
